(19) United States
(12) Patent Application Publication
(10) Pub. No.: US 2026/0159593 A1
(43) Pub. Date: Jun. 11, 2026

(54) TREATMENT OF HEMOLYTIC UREMIC SYNDROMES

(71) Applicant: The General Hospital Corporation, Boston, MA (US)

(72) Inventors: Eric F. Grabowski, Wellesley, MA (US); Gregory L. Stahl, Urbana, OH (US); Julie R. Ingelfinger, Cambridge, MA (US)

(21) Appl. No.: 18/706,658

(22) PCT Filed: Nov. 1, 2022
(86) PCT No.: PCT/US2022/048523

§ 371 Date: May 1, 2024

Related U.S. Application Data
(60) Provisional application No. 63/274,175, filed on Nov. 1, 2021.
(60) Provisional application No. 63/316,441, filed on Mar. 4, 2022.

(51) Int. Cl. C07K 16/28; A61K 39/00; A61P 7/00; C07K 16/36

(52) U.S. Cl. CPC C07K 16/2851; A61P 7/00; C07K 16/36; A61K 2039/507

(57) ABSTRACT

Provided herein are methods of treating a thrombotic microangiopathy (TMA) in a subject, comprising administering to the subject a therapeutically effective amount of an antibody that binds to and inhibits MBL2 and an antibody that binds to and inhibits FXIa. In some embodiments, the antibody that binds to and inhibits MBL2 is 3F8 or an analogue thereof, and/or, the antibody that binds to and inhibits FXIa is 3G3 or an analogue thereof.

### CLAIM OF PRIORITY

This application claims the benefit of U.S. Provisional Application Ser. No. 63/274,175, filed on Nov. 1, 2021, and 63/316,441, filed on Mar. 4, 2022. The entire contents of the foregoing are incorporated herein by reference.

### FEDERALLY SPONSORED RESEARCH OR DEVELOPMENT

This invention was made with Government support under Grant No. DK117317 awarded by the National Institutes of Health. The Government has certain rights in the invention.

### TECHNICAL FIELD

Provided herein are methods of treating a thrombotic microangiopathy (TMA) in a subject, comprising administering to the subject a therapeutically effective amount of an antibody that binds to and inhibits MBL2 plus an antibody that binds to and inhibits FXIa. In some embodiments, the antibody that binds to and inhibits MBL2 is 3F8 or an analogue thereof, and/or, the antibody that binds to and inhibits FXIa is 3G3 or an analogue thereof.

### BACKGROUND

Enterohemorrhagic hemolytic uremic syndrome (eHUS), triggered by Shiga toxin (STX), is a thrombotic microangiopathy that is a major cause of acute kidney injury (AKI) in children (1-2). eHUS is characterized by hemolytic anemia, thrombocytopenia, and AKI and can lead to injury of other organs (e.g., stroke with cortical blindness, lung injury requiring intubation and ventilatory support) and death. Severely affected children require hospitalization and ˜65% require dialysis (450-750 cases/year in the US); 3-5% have seizures and subsequent neurocognitive deficits from cerebral ischemic events. Up to 30% develop other organ system failure, with death occurring in 3-5%. Long-term outcomes include chronic kidney disease, hypertension, insulin-dependent diabetes mellitus, and neurocognitive impairment. Currently only supportive care is available, as no therapeutic agent has been approved.

### SUMMARY

Provided herein are methods for treating disorders in which complement activation and coagulation play significant roles. The methods can include administering to a patient therapeutically effective amounts of a combination of therapies: (i) that inhibits the complement pathway and (ii) that inhibits the coagulation pathway; in some embodiments, the methods include administration of monoclonal antibodies that inhibit MBL2 (to inhibit the lectin pathway of complement) and FXIa (to inhibit the contact pathway of coagulation).

Provided herein are methods for treating a thrombotic microangiopathy (TMA) in a subject (a mammalian subject, preferably a human subject), the method comprising administering to the subject a therapeutically effective amount of an antibody that binds to and inhibits MBL2 and an antibody that binds to and inhibits FXIa. An antibody that binds to and inhibits MBL2 and an antibody that binds to and inhibits FXIa, for use in a method of treating a thrombotic microangiopathy (TMA) in a subject. In some embodiments, the methods comprise administering a pharmaceutical composition that comprises both an antibody that binds to and inhibits MBL2 and an antibody that binds to and inhibits FXIa in a single composition. In some embodiments, the methods comprise administering a first pharmaceutical composition that comprises an antibody binds to and that inhibits MBL2 and a second pharmaceutical composition that comprises an antibody that binds to and inhibits FXIa, in a separate composition. The antibodies can be administered substantially concurrently, or consecutively in any order.

In some embodiments, the antibody that binds to and inhibits MBL2 is 3F8 or an analogue thereof, and/or, the antibody that binds to and inhibits FXIa is 3G3 or an analogue thereof.

In some embodiments, the antibodies are human or humanized.

In some embodiments, the subject has enterohemorrhagic hemolytic uremic syndrome (eHUS); atypical HUS (aHUS); myocardial ischemic reperfusion injury; renal ischemic reperfusion injury; stroke; eclampsia or pre-eclampsia; catastrophic antiphospholipid antibody syndrome; complement-related renal injury; hematopoietic stem cell transplant thrombotic microangiopathy; paroxysmal nocturnal hemoglobinuria (PNH); disseminated intravascular coagulation (DIC); or venous or arterial thrombosis.

In some embodiments, the stroke is arterial ischemic stroke or perinatal arterial ischemic stroke.

Also provided herein are pharmaceutical compositions comprising as active agents an antibody that binds to and inhibits MBL2 and an antibody that binds to and inhibits FXIa, and a pharmaceutically acceptable carrier. In some embodiments, the antibody that inhibits MBL2 is 3F8 or an analogue thereof, and/or, the antibody that inhibits FXIa is 3G3 or an analogue thereof. In some embodiments, the antibodies are human or humanized.

Further provided herein are methods of treating a thrombotic microangiopathy in a patient by administering a combination of two monoclonal antibodies, 3F8 or one of its analogues and 3G3 or one of its analogues.

In some embodiments, the thrombotic microangiopathy (TMA) is selected from enterohemorrhagic hemolytic uremic syndrome; atypical HUS; myocardial ischemic reperfusion injury; renal ischemic reperfusion injury; arterial ischemic stroke in older children and adults; perinatal arterial ischemic stroke, eclampsia; stressful labor and delivery (e.g., a prolonged second stage of labor); catastrophic antiphospholipid antibody syndrome; hematopoietic stem cell transplant-associated TMA paroxysmal nocturnal hemoglobinuria.

In some embodiments, the 3F8 analogue is the anti-MASP-2 monoclonal antibody (Omeros Corporation) and the 3G3 analogue is BAY1213790.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Weight loss in eHUS mice following injection of STX-2, without vs. with 3F8 alone, 3G3 alone, or 3F8 plus 3G3. Phosphate buffered saline (PBS) control animal are included for comparison. Individual curves are labelled according to treatment. As can be seen, the weight loss was lessened by each antibody alone, and more so by both together.

FIGS. 2A-C. Platelets and fibrin in glomeruli of our mouse model of eHUS. These are representative animals that received STX-2 and no antibodies. 2A, Platelets stained using an A488 secondary antibody plus an anti-CD41 primary antibody. 2B, Fibrin, stained using an A647 antibody secondary antibody directed against a primary antibody that recognizes the beta chain of human fibrin. This primary antibody cross-reacts with mouse. 2C, Co-localization of platelets and fibrin in a representative glomerulus. The colocalization demonstrated that a substantial number of platelets were found in the same pixel (picture element) locations as fibrin, consistent with the presence of platelet-fibrin structures known as platelet thrombi. Such thrombi are pathognomonic of eHUS.

FIG. 3. Glomerular platelet deposition in eHUS mice, without and with antibodies. The Y-axis shows platelet deposition normalized to the mean of deposition for mice receiving STX-2. Heterogeneity in platelet deposition among mice, with 10 glomeruli analyzed for each mouse. Grey circles indicate STX-2 only, while open circles indicate STX-2 plus 3F8 and 3G3. Means for each mouse are represented by the horizontal bars.

FIG. 4. Glomerular fibrin deposition in eHUS mice, without or with antibodies. The Y-axis shows fibrin deposition normalized to the mean of deposition for mice receiving STX-2. Heterogeneity in fibrin deposition among mice, with 10 glomeruli analyzed for each mouse. Grey circles indicate STX-2 only, while open circles indicate STX-2 plus 3F8 and 3G3. Means for each mouse are represented by the horizontal bars.

FIG. 5. Glomerular platelet deposition in eHUS mice, without or with antibodies. 3F8 plus 3G3 is superior to either mAb alone in reducing glomerular platelet deposition. Individual curves are labelled according to treatment.

FIG. 6. Glomerular fibrin deposition in eHUS mice, without or with antibodies. 3F8 plus 3G3 is superior to either mAb alone in decreasing glomerular fibrin. Individual curves are labelled according to treatment.

FIG. 7. Left: Platelets. Right: Platelets stained using anti-GPIIb-IIIa (activated) and anti-CD41 (activated and non-activated). The activated and non-activated platelets were nearly 100% colocalized to the activated platelets, indicating that virtually all of the circulating platelets were activated.

FIG. 8. Platelet count vs. treatment group. The Y-axis shows platelet number density.

FIG. 9. Renal tubular injury in eHUS mice, without or with antibodies. Percent tubular injury vs. treatment group is shown.

FIG. 10. Serum NGAL levels in eHUS mice for different treatment groups. NGAL levels are in ng/ml. Treatment decreased NGAL, a marker of acute kidney injury.

### DETAILED DESCRIPTION

An effective therapy for this devastating childhood thrombotic microangiopathy is lacking. Prior efforts with agents such as eculizumab (anti-C5 mAb) therapy (5-6), the STX binding agent Synsorb Pk (7), and antibiotics (8-9) have failed (5, 10, 31). Anti-C5 therapies, used in atypical HUS in the form, for instance, of Soliris, are not effective in eHUS, as they do not act sufficiently upstream in the complement cascade. Anticoagulation therapies alone are ineffective in eHUs. Although complement activation has long been recognized in eHUS (44-46), the failure of eculizumab in eHUS suggests complement activation occurs at an earlier step than the terminal complement pathway. Further, there have been few clinical trials in eHUS. Presently, ClinicalTrials.gov lists only one active trial in eHUS: NCT05219110 (a study of the effects of hyperhydration).

Described herein are combination therapies that are believed to work by inhibiting two therapeutic targets: the mannan binding lectin-2 (MBL2) of the lectin pathway of complement (3) and clotting factor XIa (FXIa) of the contact pathway of coagulation. As shown herein, these pathways have important roles in an eHUS mouse model, demonstrated using 3F8, a mouse anti-human MBL2 (3), and 3G3, a humanized anti-FXIa (also known as AB023, ref. 4); treatment of mice with the combination was superior to treatment with either antibody alone with regard to animal survival, inhibition of platelet-fibrin thrombi in renal glomeruli, prevention of renal injury, and prevention of weight loss.

As shown herein, combination of 3F8 and 3G3 significantly ensures mouse survival to day 4, reduces glomerular platelet-fibrin deposition via IF of thin kidney sections, and reduces acute kidney injury as assessed by kidney morphologic injury scores and serum Ngal levels. The actual degree of kidney injury is almost certainly underestimated, as the nearly half of the animals receiving STX-2 that died prior to day 4 and could not be included in the day 4 kidney tissue analysis. The combination also minimized weight loss following injection of STX-2. As 3G3 does not prevent the downstream activation of clotting by factor XIa, bleeding complications are minimized [4]. The experiments using 3F8 and 3G3 alone, moreover, clearly demonstrated unexpected superiority of the antibody combination over either antibody alone.

The platelets observed in mouse glomeruli were virtually all activated, as shown by the comparison of IF images obtained with a primary anti-platelet antibody that recognizes the active conformation of GPIIb-GPIIa vs. and antibody that recognizes GPIIb only. That the platelets were all activated is likely a consequence of STX-2, known to directly bind to and activate platelets (23).

The combination therapy affords a novel approach to the treatment of diseases including enterohemorrhagic HUS in children. This combination therapy, with both antibodies, may provide the first specific and effective therapy for this devastating disease.

### Methods of Treatment

Provided herein are methods for treating disorders in which complement activation and coagulation play significant roles. Such disorders include, without limitation, the following: thrombotic microangiopathies (TMAs) such as enterohemorrhagic hemolytic uremic syndrome (eHUS) (1); atypical HUS (aHUS; due to deficiencies and/or mutations in complement regulatory proteins, 31); myocardial ischemic reperfusion injury (33); renal ischemic reperfusion injury; stroke (32), e.g., arterial ischemic stroke in older children and adults (17) and perinatal arterial ischemic stroke, in which stroke triggers include maternal pre-eclampsia and a stressful labor and delivery (e.g., a prolonged second stage of labor); eclampsia and pre-eclampsia (34, 42; due to suspected deficiencies and/or mutations in complement regulatory proteins); catastrophic antiphospholipid antibody syndrome (catastrophic APS, 48); MBL2-related renal injury (35), hematopoietic stem cell transplant thrombotic microangiopathy (36) and paroxysmal nocturnal hemoglobinuria (PNH), which carries a marked thrombosis risk (47); disseminated intravascular coagulation (DIC) (18); and venous or arterial thrombosis (37, 43).

The methods can include administering to a patient therapeutically effective amounts of a combination of therapies: (i) that inhibits the complement pathway and (ii) that inhibits the coagulation pathway; in some embodiments, the methods include administering monoclonal antibodies that inhibit MBL2 (inhibiting the lectin pathway of complement) and FXIa (inhibiting the contact pathway of coagulation). In some embodiments, the methods include administering a pharmaceutical composition that includes both an antibody that inhibits MBL2 and an antibody that inhibits FXIa in a single composition. Alternatively, the methods can include administering an antibody that inhibits MBL2 and an antibody that inhibits FXIa in separate compositions.

As used herein, to “treat” means to ameliorate at least one symptom of the disorder described herein. Thus, a treatment can result in one or more of preservation of normal kidney function, protection against ischemic injury to the kidneys, and potentially also protection against ischemic injury to the brain, pancreas, and lungs, and/or confer improvement in quality of life for the expected lifetime of an affected child (7-to-8 more decades) by reducing risk of long term effects, particularly for the 10% of children who subsequently develop chronic kidney disease (CKD), chronic arterial hypertension, neurological impairment, and/or diabetes mellitus (10).

The present data demonstrate that the antibody combination retains efficacy even after a delay of 24 hours from the time of injection of STX2 to the administration of the antibody combination, thus benefitting patients who present with STX infection. As the dose administered to our mice was associated with a ten-fold greater mortality than that seen in affected children and as antibody half-lives are three-fold longer in humans than in mice, the above time interval could be substantially longer than 24 hours. This would allow sufficient time for clinical recognition of eHUS in a child and its subsequent administration based on the usual clinical triad of thrombocytopenia, thrombotic microangiopathy, and acute kidney injury. Expected potential benefits would be 1) the elimination of a substantial proportion of the necessary courses of dialysis, or at least a significantly shortened duration of these courses of dialysis; 2) decrease in hospital ICU days; 3) better preserved long-term kidney function; 4) preservation of brain, pancreatic, lung, and other organ function; and 5) prevention of death. This approach to treatment would provide a successful alternative to supportive care (which includes dialysis) and would likely become standard-of-care for any child who presents with clinical eHUS. These benefits would extend also to adult patients who develop eHUS, since outbreaks of eHUS, can on occasion affect adults, as happened in an outbreak in northern Germany in 2011 (30)

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Exemplary doses are provided below based on an FDA guidance document (19). These doses are exemplary only and the skilled physician can decide on actual dosing based on such factors as age, body weight, and disease severity. We note that in view of the absence to date of a specific and effective treatment for eHUS, any dose safety and dose efficacy trial needs only to compare treatment to supportive care. Exemplary conversions are as follows:

| | Dose in mice | Dose in 20 kg | Dose in 20 kg |
|---|---|---|---|
| Antibody | (μg/g IP) | human (mg/m² IV) | human (mg/kg IV) |
| 3F8 | 30 | 750 | 25 |
| 3G3 | 5 | 125 | 4.2 |

Thus, an exemplary dosage range for 3F8 would be 10-50 mg/kg IV in a 20 kg child, and 15-75 mg/kg IV in an adult, using Table 7 of the FDA guidelines cited, and the range for 3G3 would be 2-10 mg/kg IV in a 20 kg child, and 3-15 mg/kg in an adult using the same guidelines.

### Antibodies

In some embodiments, the antibody that inhibits MBL2 is 3F8 or an analogue thereof (Collard et al., Am J Pathol. 2000 May; 156 (5): 1549-1556). 3F8 that has been shown to reduce Shiga toxin-induced renal injury in STEC HUS. See, Ozaki et al, Kidney International 90 (4); October 2016; 774-782. 3F8 has been shown to bind within the carbohydrate recognition domain (CRD) of MBL, making a conformational change in MBL that inhibits ligand binding and thus inhibits MBL-dependent complement activation (Collard et al., 2000). 3F8 inhibits MBL2 and its associated proteases, MASP-1 and MASP-2, which activate the clotting system, specifically factor Xa and factor IIa (thrombin). In some embodiments, a humanized version of the 3F8 antibody is used.

In some embodiments, the antibody that inhibits FXIa is 3G3 or an analogue thereof. 3G3 (also known as AXIMAb or AB023 or Xisomab 3G3) is a humanized version of the murine monoclonal 14E11 (19). 3G3 blocked activation by FXIIa and reduced activation of coagulation and fibrinogen and platelet consumption, which decreased inflammation and prevented organ failure in baboons challenged with S. aureus. See, Silasi et al, Blood Advances 3 (4): 658-669 (2018). See also WO2020154234.

By “antibody” is meant a monoclonal antibody or derivative thereof that exhibits substantially equivalent functionality or biological activity as either 3F8 or 3G3, for example by inhibiting the complement pathway or by inhibiting the coagulation pathway. Without wishing to be bound by theory, it is proposed that inhibiting both of these pathways results in the efficacious therapeutic benefit seen in vivo in the murine model described below.

The term “antibody” as used herein refers to an immunoglobulin molecule or an antigen-binding portion thereof. Examples of antigen-binding portions of immunoglobulin molecules include F(ab) and F(ab′)₂ fragments, which retain the ability to bind antigen. The antibodies can be monoclonal, recombinant, chimeric, de-immunized or humanized, fully human, non-human, (e.g., murine), or single chain antibody versions of the antibody. In some embodiments, the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

### Pharmaceutical Compositions and Methods of Administration

The methods and compositions described herein include the use of pharmaceutical compositions comprising or consisting of an antibody that inhibits MBL2 (e.g., 3F8 or an analogue thereof) and/or an antibody that inhibits FXIa (e.g., 3G3 or an analogue thereof) as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Methods

The following materials and methods were used in the Example below.

Mouse model. This mouse model replicates key features of eHUS (thrombocytopenia, hemolytic anemia, and kidney injury) while also being a knock-out of mouse Mbl2 and 2 and with knock-in of human MBL2 to accommodate our anti-human MBL2 mAb (3F8, ref. 3). There is no anti-mouse Mbl 1/2, as most species (except humans and chickens) have two functional MBLs (22). Modifications from the original model (3) were made such that 40-45% of untreated mice expire by day 4 (hour 96) following STX-2 injection, while also mice treated with 3F8, 3G3, or the combination all survived to day 4 (with the exception of one animal treated with 3F8 alone) to permit tissue assessments of kidney glomerular platelet-fibrin deposition and renal injury, and blood sampling for blood counts and measures of kidney function. Mice not receiving one or both monoclonal antibodies received appropriate isotype control antibodies. Approximately 24 experiments using a total of 140 test and control animals were performed at doses of STX-2 of 125, 150, 175, 200, 250, 300, and 350 μg/kg ip, with two different lots of STX-2, and for observation periods prior to planned sacrifice of 72 and 96 hours. The present model used 300 pg/g of a “standard” lot of STX-2 and an observation period of 96 hours, at which time all surviving mice were euthanized with 5% isoflurane, have blood samples drawn via cardiac puncture, and have kidney tissues, both fresh frozen and formalin-fixed, collected for histology. Fractional weight loss over four days was measured daily, and observations made of animal activity, posture and coat quality.

Fibrin and platelet deposition in glomeruli. We used thin sections (5 microns) of kidney tissue fresh frozen in OTC to examine fibrin and platelet deposition, a key thrombotic endpoint. Images were acquired at 10× and 20× using digital epifluorescence video microscopy (>1.4×10⁶ pixels per image), and analyzed via MetaMorph Premier software for total glomerular pixels occupied by fibrin or platelets, and total fibrin and total platelet fluorescence intensity, measures of fibrin amount and platelet thrombus volume (platelet number), respectively. Platelets were co-localized to fibrin (composite images), consistent with the presence of platelet-fibrin thrombi.

Kidney injury assessment. Mouse kidney injury following injection with STX-2, without or with treatment, was assessed using routine histologic sections stained with hematoxylin and eosin (H&E) and periodic-acid Schiff (PAS). Injury was evaluated in all kidney compartments. The extent of tubular injury was quantified as the mean of percent tubular injury in 5 random medium power (20×) fields. For each animal treated, we also collected serum samples for measurement of serum neutrophil gelatinase-associated lipocalin (NGAL) using a specific ELISA (Sigma Hemoglobin Assay Kit Catalog #MAK115-1KT; see also Devarajan et al., Scand J Clin Lab Invest Suppl. 2008; 241:89-94).

Platelet counts. Complete blood counts in blood collected into EDTA were determined in our animal facility using a Coulter Counter. These counts were compared to counts obtained previously (3) in control MBL2KI mice identical to those used in the present study.

Statistics. Animal weights, NGAL values, and platelet counts for the treatment groups, which followed normal distributions, were compared using ANOVA, followed by the Tukey test for comparisons (two-sided). Values for platelet and fibrin deposition were calculated as the mean total intensity for image pixels present in circular regions of interest centered over and just covering individual glomeruli (average diameter approximately 70 μm). Mean total intensity measures the total number of platelets or total relative quantity of fibrin for each glomerulus. For each animal, the results for at least 30 glomeruli were averaged and normalized by the mean of control values to minimize the effect of variations in baseline values among mice. Treatment groups were then compared using the non-parametric Kruskal-Wallis test followed by the Mann-Whitney test for comparisons. Renal histologic injury scores were also analyzed by the Kruskal-Wallis and Mann-Whitney tests.

### Example 1. Specific and Effective Therapy for the Enterohemorrhagic Hemolytic Uremic Syndrome (eHUS): Blockade of the Lectin Pathway of Complement and the Contact Pathway of Coagulation

The murine model of enterohemorrhagic hemolytic uremic syndrome (eHUS) used herein was developed using a mouse humanized C57BL/6 MBL-2 model (MBL-2^{+/+}Mbl-1^{-/-}Mbl-2^{-/-}; aka KI mice) (3). Clotting parameters and platelets in the Mbl-null mice are similar to those of wild type mice, indicating that coagulation itself is not defective in Mbl deficiency. This mouse model has been used in studies of sepsis, myocardial reperfusion injury, and, most recently, eHUS.

Mice treated with anti-MBL2 (3F8, 30 μg/g) in combination with anti-FXIa (3G3, also known as AB023 (4); 5 μg/g) at the time of exposure to STX-2 demonstrated 100% survival (31/31) with combination monoclonal antibody treatment vs. 43% survival (24/56) without. Survival with either 3F8 or 3G3 alone was 96% (27/28). All deaths occurred between days 3 and 4.

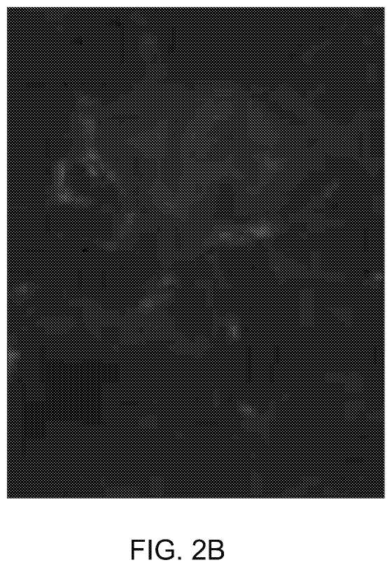

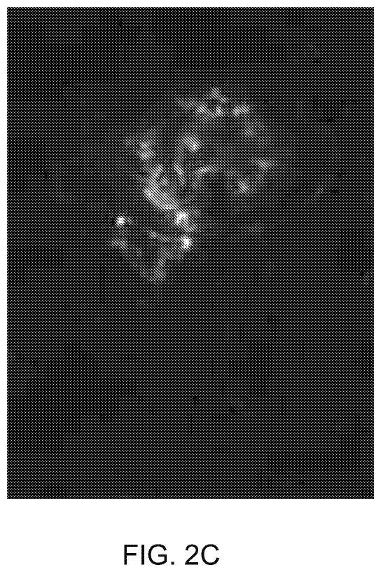

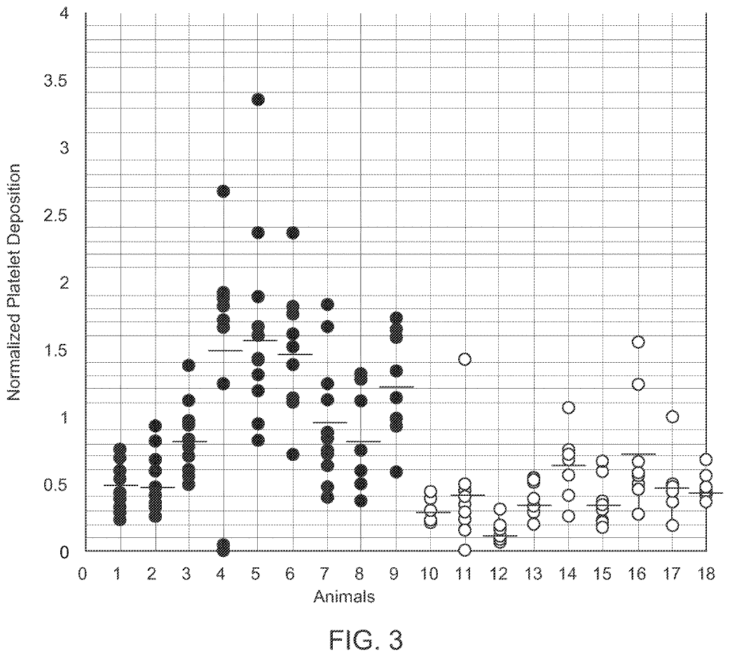

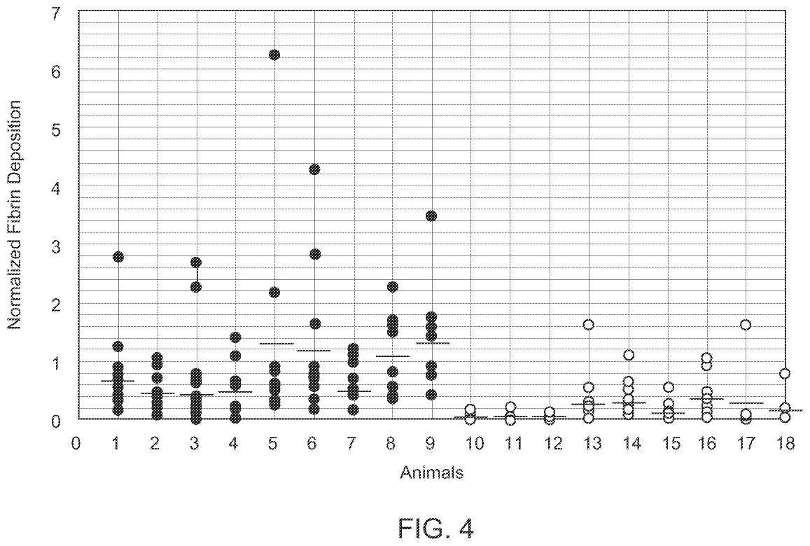

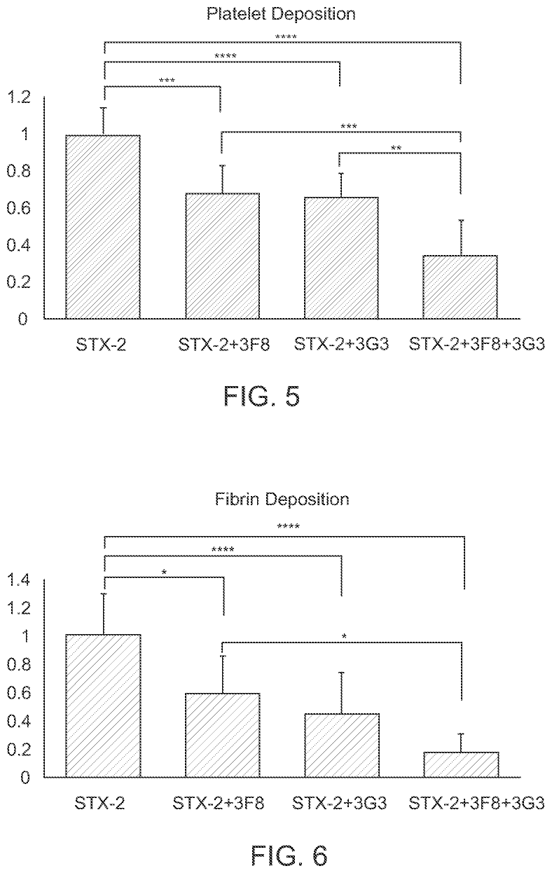

With regard to platelet and fibrin deposition, the FIGS. 2A-B show platelets and fibrin in mouse kidney sections by immunofluorescence (IF) for one glomerulus, while FIG. 2C shows co-localization of platelets to fibrin (composite image), characteristic of platelet-fibrin thrombi. FIG. 3 shows the heterogeneity in platelet deposition in all glomeruli studied for representative mice, with means for each mouse represented by the horizontal bars. Heterogeneity in fibrin deposition was similar, as depicted in FIG. 4. Using the means for 10 glomeruli per mouse, significant reductions in platelet and fibrin deposition are shown in FIGS. 5 and 6 for each different treatment. The combination of 3F8 and 3G3 yielded a 63.4±1.7% reduction (p<0.0001) in platelet deposition and an 80.5±1.3% reduction (p<0.0001) in glomeruli fibrin deposition, as compared to mice receiving isotype control antibodies. Moreover, the antibody combination was clearly superior to either 3F8 or 3G3 alone (FIGS. 5 and 6).

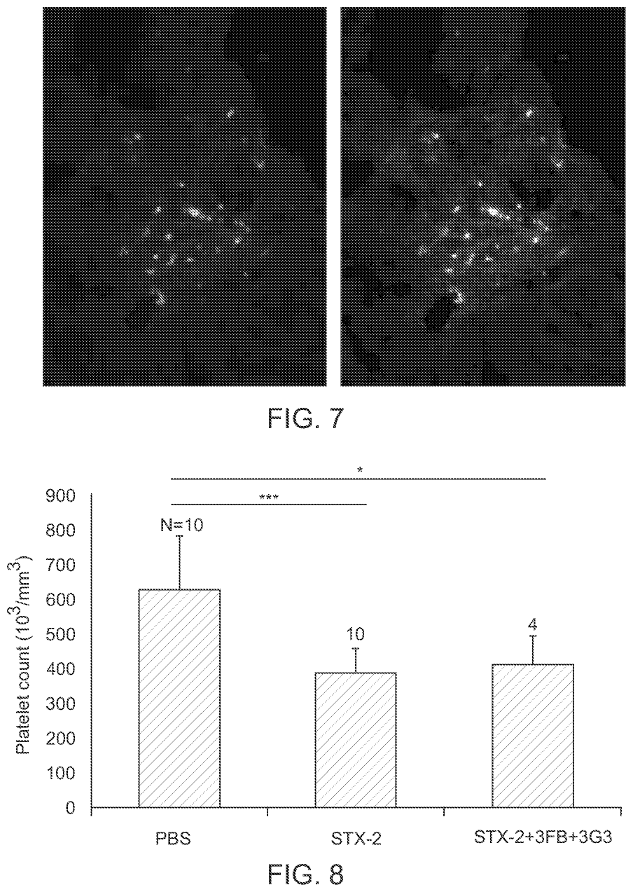

Parallel assessment of activated platelets using an antibody directed against platelet GPIIb-GPIIIa, that recognizes activated platelets, and GPIIb alone (anti-CD41), that recognizes activated and non-activated platelets, shows that virtually all (>95%) of the glomerular platelets are activated (FIG. 7). A total fluorescence intensity calibration from images of single mouse platelets indicates that there are 80 to 120 platelets per glomerular section, in agreement with a theoretical calculated value of 83.

At the time the mice were euthanized, platelet counts for mice receiving STX2 were 385,000±74,000 (mean±SD, N=10), which is significantly lower (p=0.00051) than the 625,000±160,000 for control mice receiving PBS (FIG. 8). Mice receiving STX-2 together with 3F8 and 3G3 also had platelet counts significantly lower (p=0.018) than those receiving PBS.

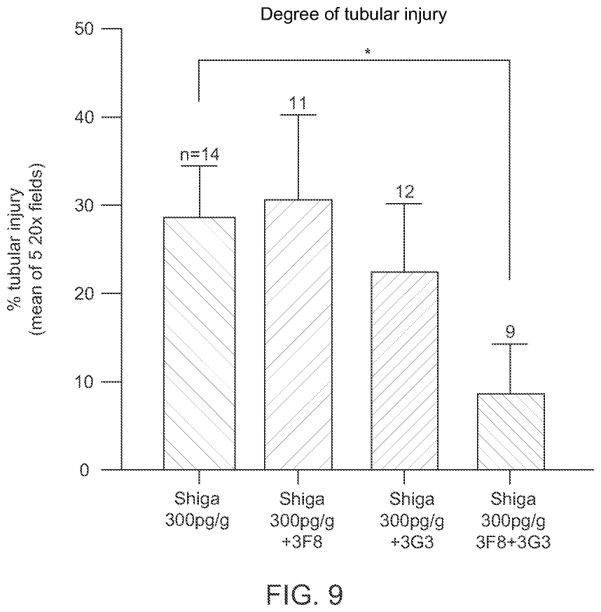

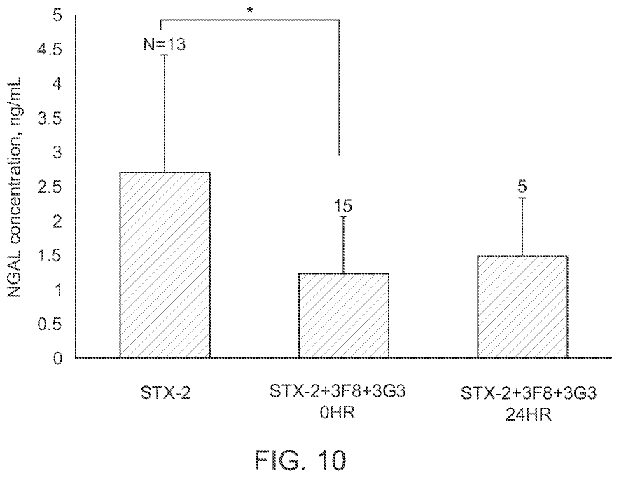

Glomeruli in mice receiving STX-2 appeared congested at the time of euthanasia, regardless of antibody treatment. Treatment-related differences in glomerular injury were not identifiable, possibly because such differences cannot be discriminated by brightfield microscopy. Acute kidney injury was therefore based largely on percent tubular injury, averaged for five fields of view at 10×. Percent tubular injury was significantly reduced (p<0.05) with the antibody combination vs. no antibody treatment (FIG. 9). No significant reductions were found with either 3F8 or 3G3 alone. At the same time, values for NGAL, a sensitive marker of acute kidney injury, were reduced (p=0.0058) by 57% with the antibody combination vs. no treatment (FIG. 10). Although not statistically significant, there was a trend for a reduction in NGAL when the antibody combination was given at hour 24.

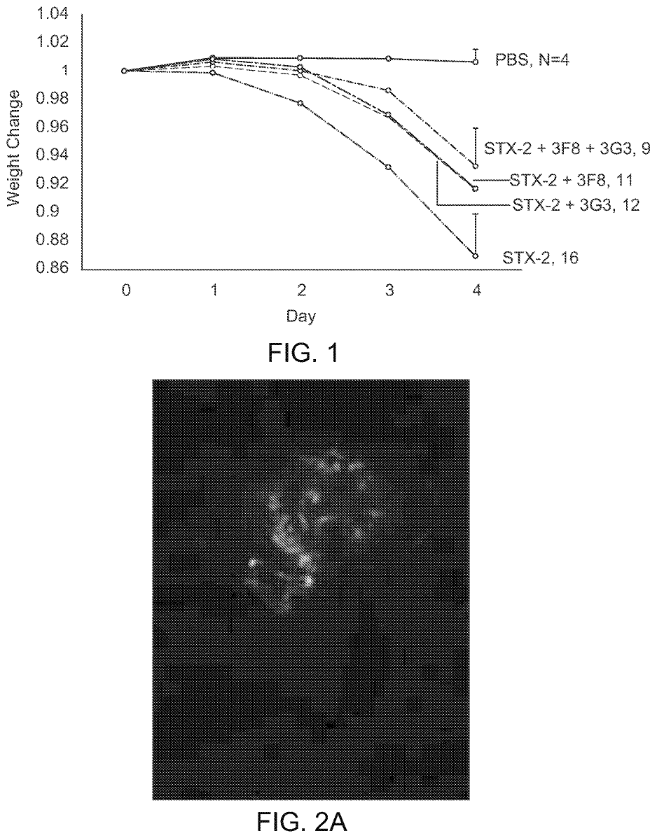

Initial animal weight was better preserved with 3F8 plus 3G3 given at the same time as injection of STX-2 (FIG. 1 and Table 1). Day 4 percent weight losses with combination antibody treatment were 6.8% of baseline, vs. 13.1% with no treatment (difference significant with p=0.000046), and 8.3 and 8.4% with either 3G3 or 3F8 alone, respectively (FIGS. 1 and Table 1; differences significant with p=0.00097 and 0.0015, respectively). Day 3 percent weight losses were also significantly reduced with combination antibody treatment, and with either antibody alone (Table 3). The combination antibody treatment was superior at day 3 to either antibody alone. Percent weight loss at day 4 was also less when the antibody combination was given at 24 hours (p<0.049, Table 2), and percent weight loss at day 3 also less when the antibody combination was administered at 48 hours (p<0.020) or 24 hours (p<0.00040) (Table 4). For day 4 weights, significance at hour 48 was likely not achieved owing to the death of many animals receiving STX-2 prior to day 4 and their inclusion in the weight analysis not possible. Nonetheless, there still was a suggestion of better weight preservation at 48 hours (Table 2, with a projected significance of p<0.05 with an N of 14). Absolute weight losses (vs. percent weight changes) were significantly lessened by the antibody combination given at time zero, although the level of significance was lower (p=0.0084). Animals receiving STX-2 without any treatment tended to be moribund and with poor coat quality by day 3.

TABLE 1
Day 4 (hour 96) fractional weight loss after injection
of STX-2 at hour zero, and with treatment hour
zero. PBS control is shown for comparison.

| Treatment | N | Mean ± SD | p value |
|---|---|---|---|
| STX2 | 16 | 0.869 ± 0.029 | |
| STX2 + 3F8 + 3G3 | 9 | 0.932 ± 0.026 | 0.0002599 |
| STX2 + 3F8 | 11 | 0.916 ± 0.036 | 0.003261 |
| STX2 + 3G3 | 12 | 0.917 ± 0.027 | 0.0003461 |
| PBS | 4 | 1.006 ± 0.004 | 0.0004128 |

TABLE 2
Day 4 (hour 96) fractional weight loss after injection
of STX-2 at hour zero, and with the combination
of 3F8 and 3G3 at hours zero, 24, and 48.

| Treatment | N | Mean ± SD | p value |
|---|---|---|---|
| STX2 | 14 | 0.847 ± 0.03490 | |
| STX2 + 3F8 + 3G3 Hr 0 | 8 | 0.910 ± 0.02698 | 0.0003 |
| STX2 + 3F8 + 3G3 Hr 24 | 8 | 0.883 ± 0.03043 | 0.0222 |
| STX2 + 3F8 + 3G3 Hr 48 | 6 | 0.868 ± 0.02067 | 0.1782 |

TABLE 3
Day 3 (hour 72) fractional weight losses after injection
of STX-2 at hour zero, and with treatment at hour
zero. PBS control is shown for comparison.

| Treatment | N | Mean ± SD | p value |
|---|---|---|---|
| STX2 | 16 | 0.931 ± 0.026 | |
| STX2 + 3F8 + 3G3 | 9 | 0.986 ± 0.019 | <0.0001 |
| STX2 + 3F8 | 11 | 0.969 ± 0.050 | 0.0011 |
| STX2 + 3G3 | 12 | 0.967 ± 0.017 | 0.0005 |
| PBS | 4 | 1.008 ± 0.004 | 0.0004 |

TABLE 4
Day 3 (hour 72) fractional weight losses after injection
of STX-2 at hour zero, and with the combination
of 3F8 and 3G3 at hours zero, 24, and 48.

| Treatment | N | Mean ± SD | p value |
|---|---|---|---|
| STX2 | 14 | 0.905 ± 0.032 | |
| STX2 + 3F8 + 3G3 Hr 0 | 8 | 0.969 ± 0.026 | 0.0002 |
| STX2 + 3F8 + 3G3 Hr 24 | 8 | 0.958 ± 0.018 | 0.0004 |
| STX2 + 3F8 + 3G3 Hr 48 | 6 | 0.946 ± 0.028 | 0.020 |

### REFERENCES

1. Tesh V L. Induction of apoptosis by Shiga toxins. Future Microbiol. 2010 March; 5 (3): 431-53.
2. Koster F T, Boonpucknavig V, Sujaho S, Gilman R H, Rahaman M M. Renal histopathology in the hemolytic-uremic syndrome following shigellosis. Clin Nephrol 1984; 21 (2): 126-33.
3 Ozaki M, Kang Y, Tan Y S, Pavlov V I, Liu B, Boyle D C, Kushak R I, Skjoedt M O, Grabowski E F, Taira Y, Stahl G L. Human mannose-binding lectin inhibitor prevents Shiga toxin-induced renal injury. Kidney Int. 2016 October; 90 (4): 774-82.
4. Lorentz C U, Verbout N G, Wallisch M, Hagen M W, Shatzel J J, Olson S R, Puy C, Hinds M T, McCarty O J T, Gailani D, Gruber A, and Tucker E I. The contact activation inhibitor and factor XI antibody, AB023, produces safe, dose-dependent anticoagulation in a phase 1 first-in-human trial. Arterioscler Thromb Vasc Biol. 2019; 39:799-809.
5. Menne J, Nitschke M, Stingele R, Abu-Tair M, Beneke J, Bramstedt J, et al. Validation of treatment strategies for enterohaemorrhagic Escherichia coli O104:H4 induced haemolytic uraemic syndrome: case control study. BMJ 2012; 345:e4565.
6. Keir L S and Langman C B. Complement and the kidney in the setting of Shiga-toxin hemolytic uremic syndrome, organ transplantation, and C3 glomerulonephritis. Transfus Apher Sci. 2016 April; 54 (2): 203-
7. Howard Trachtman 1, Avital Cnaan, Erica Christen, Kathleen Gibbs, Sanyi Zhao, David W K Acheson, Robert Weiss, Frederick J Kaskel, Adrian Spitzer, Gladys H Hirschman, Investigators of the HUS-SYNSORB Pk Multicenter Clinical Trial. Effect of an oral Shiga toxin-binding agent on diarrhea-associated hemolytic uremic syndrome in children: a randomized controlled trial. JAMA 2003 Sep. 10; 290 (10): 1337-44.
8. Safdar N, Said A, Gangnon R E, Maki D G. Risk of hemolytic uremic syndrome after antibiotic treatment of Escherichia coli 0157:H7 enteritis: a meta-analysis. JAMA. 2002; 288:996-1001.
9. Wong C S, Jelacic S, Habeeb R L, Watkins S L, Tarr P I. The risk of the hemolytic-uremic syndrome after antibiotic treatment of Escherichia coli 0157:H7 infections. N Engl J Med. 2000; 342:1930-6.
10. Bitzan M, Schaefer F, and Didier R. Treatment of Typical (Enteropathic) Hemolytic Uremic Syndrome. Semin. Thromb. Hemost. 2010; 36:594-610.
11. Conway E M. Complement-coagulation connections. Blood Coagul Fibrinolysis. 2018 April; 29 (3): 243-251
12. Tedesco F, Pausa M, Nardon E, et al. The cytolytically inactive terminal complement complex activates endothelial cells to express adhesion molecules and tissue factor procoagulant activity. J Exp Med. 1997 May 5; 185 (9): 1619-27.
13. Amara U, Flierl M A, Rittirsch D, Klos A, Chen H, Acker B, et al. Molecular intercommunication between the complement and coagulation systems. J Immunol. 2010; 185:5628-36.
14. Morigi M, Galbusera M, Gastoldi S, Locatelli M, Buelli S, Pezzotta A, Pagani C, Noris M, Gobbi M, Stravalaci M, Rottoli D, Tedesco F, Remuzzi G, and Zoja C. Alternative pathway activation of complement by Shiga toxin promotes exuberant C3a formation that triggers microvascular thrombosis. J. Immunol. 2011; 187:172-80.
15. La Bonte L R, Pavlov V I, Tan Y S, et al. Mannose-binding lectin-associated serine protease-1 is a significant contributor to coagulation in a murine model of occlusive thrombosis. J Immunol. 2012 Jan. 15; 188 (2): 885-91.
16. L R, Pavlov V I, Tan Y S, Takahashi K, Takahashi M, Banda N K, Zou C, Fujita T, Stahl G L. Mannose-binding lectin-associated serine protease-1 is a significant contributor to coagulation in a murine model of occlusive thrombosis. J Immunol. 2012; 188:885-91.
17. Takahashi K, Chang W-C, Takahashi M, Pavlov V, Ishida Y, La Bonte L, Shi L, Fujita T, Stahl G L*, Van Cott E M* (*co-senior authors). Mannose-binding lectin and its associated proteases (MASPs) mediate coagulation and its deficiency is a risk factor in developing complications from infection, including disseminated intravascular coagulation. Immunobiology 2011; 216:96-102.
18. Krarup A, Wallis r, Presanis J S, Gal P, and Sim R B. Simultaneous activation of complement and coagulation by MBL-associated serine protease 2. PLOS One. 2007; 2: e623.
19. Puy C, Pang J, Reitsma S E, Lorentz C U, Tucker E I, Gailani D, Gruber A, Lupu F, and McCarty O J T. Cross-Talk between the Complement Pathway and the Contact Activation System of Coagulation: Activated Factor XI Neutralizes Complement Factor H. Journal of Immunology. 2021; 206:1784-1702.
20. Schmaier A H. Antithrombotic potential of the contact activation pathway. Curr. Opin. Hematol. 2016; 23:445-452.
21. Puy C, Tucker E I, Matafonov A, Cheng Q, Zientek K D, Gailani D, Gruber A, and McCarty O J T. Activated factor XI increases the procoagulant activity of the extrinsic pathway by inactivating tissue factor pathway inhibitor. Blood 2015; 26:1488-96.
22. Kjærup R M, Norup L R, Skjødt K, Dalgaard T S, Juul-Madsen H R. Chicken mannose-binding lectin (MBL) gene variants with influence on MBL serum concentrations. Immunogenetics. 2013; 65:461-71.
23. Ghosh A, Polanowski-Grabowska R K, Fujii J, Obrig T, and Geard A R L. Shiga toxin binds to activated platelets. Journal Thrombosis and Haemostasis 2004; 2:499-506.
24. Chan Y S and Ng T B. Shiga toxins: from structure and mechanism to applications. Appl Microbiol Biotechnol. 2016; 100:1597-1610.
25. Khine A A and Lingwood C A. Capping and Receptor-Mediated Endocytosis of Cell-Bound Verotoxin (Shiga-Like Toxin) 1: Chemical Identification of an Amino Acid in the B Subunit Necessary for Efficient Receptor Glycolipid Binding and Cellular Internalization. J Cell Physiol 1994; 161:319-332.
26. Samuel J E, Perera L P, Ward S, O'Brien A D, Ginsburg V, and Krivan H C. Comparison of the Glycolipid Receptor Specificities of Shiga-Like Toxin Type II and Shiga-Like Toxin Type II Variants. Infectious Immunology 1990; 58:611-618.
27. Zoja C, Locatelli M, Pagani C, Corna D, Zanchi C, Isermann B, Remuzzi G, Conway E M, and Noris M. Lack of the lectin-like domain of thrombomodulin lessens worsens Shiga Toxin-associated hemolytic uremic syndrome in mice. J Immunol Oct. 1, 2012, 189 (7) 3661-3668.
28. Takata S, Sawa Y, Uchiyama T, Ishikawa H. Expression of toll-like receptor 4 in glomerular endothelial cells under diabetic conditions. Acta Histochem Cytochem 2013; 46:35-42
29. Torgersen M L, Engedal N, Pederson A G, Husebye H, Espevik T, and Sandvig K. Toll-like receptor 4 facilitates binding of Shiga toxin to colon carcinoma and primary umbilical vein endothelial cells. FEMS Immunol. Med. Microbiol. 2011; 61:63-75.
30. Eisen D P. Mannose-binding lectin deficiency and respiratory tract infection. J Innate Immun. 2010; 2 (2): 114-22.
31. Buchholz U, Bernard H, Werber D, Böhmer MM, Remschmidt C, Wilking H, Delere Y, an der Heiden M, Adlhoch C, Dreesman J, Ehlers J, Ethelberg S. German Outbreak of Escherichia coli 0104: H4 Associated with Sprouts. N Engl J Med 2011; 365:1763-1770
32. Bernabeu A I A, Escribano T C, Vilarino M C. Atypical Hemolytic Uremic Syndrome: New Challenges in the Complement Blockage Era. Nephron 2020; 144:637-549.
33. de la Rosa X, Cervera A, Kristoffersen A K, Valdes C P, Varma H M, Justicia C, Durduran T, Chamorro A, and Planas A M. Mannose-binding lectin promotes local microvascular thrombosis after transient brain ischemia in mice. Stroke 2014; 45:1453-9.
34. Pavlov V I, Tan Y S, McClure E E, La Bonte L R, Zou C, Gorsuch W B, Stahl G L. Human mannose-binding lectin inhibitor prevents myocardial injury and arterial thrombogenesis in a novel animal model. American Journal of Pathology 2015; 185 (2): 347-55.
35. Qing X, Redecha P B, Burmeister M A, Tomlinson S, D'Agati V D, Davisson R L, Salmon J E. Targeted inhibition of complement activation prevents features of preeclampsia in mice. Kidney International 2011; 79:331-339.
36. van der Pol P, Schlagwein N, van Gijlswijk D J, et al. Mannan-binding lectin mediates renal ischemia/reperfusion injury independent of complement activation. Am J Transplant. 2012 April; 12 (4): 877-87.
37. Jodele S, Licht C, Goebel J, Dixon B P, Zhang K, Sivakumaran T A, Davies S M, Pluthero F G, Lu L, and Laskin B L. Abnormalities in the alternative pathway of complement in children with hematopoietic stem cell transplant-associated thrombotic microangiopathy. Blood 2013; 122:2003-2007
38. Subramanian S, Jurk K, Hobohm L, et al. Distinct Contributions of Complement Factors to Platelet Activation and Fibrin Formation in Venous Thrombus Development. Blood. 2017; 129:2291-2302.
39. Guidance for Industry. E11 Clinical Investigation of Medicinal Products in the Pediatric Population. U.S. Department of Health and Human Services. Food and Drug Administration. Center for Drug Evaluation and Research (CDER). Center for Biologics Evaluation and Research (CBER). ICH. December 2000.
40. Lu R M, Hwang Y C, Liu I J, Lee C C. Tsai H Z, Li H J, and Wu H C. Development of Therapeutic Antibodies for the Treatment of Diseases. J. Biomed. Sci. 2020; 27:1.
41. Khantasup K, Chantima W, Sangma C, Poomputsa K, and Dharakul T. Design and Generation of Humanized Single-chain Fv Derived from Mouse Hybridoma for Potential. In, Monoclonal Antibodies in Immunodiagnosis and Immunotherapy, Mary Ann Liebert, Inc. 2015; 34:404-417.
42. Guidance for Industry: Estimating the maximum safe starting dose in initial clinical trials for therapeutics in adult healthy volunteers. (Available online.) US Department of Health and Human Services, Food and Drug Administration. Center for Drug Evaluation and Research (CDER). Jul. 6, 2005. Table 1, p. 7.
43. Xu Y, Brooke A. Langevin B A, Zhou H, and Xu Z. Model-Aided Adults-to-Children Pharmacokinetics Extrapolation and Empirical Body Size-Based Dosing Exploration for Therapeutic Monoclonal Antibodies—Is Allometry a Reasonable Choice? J. Clin. Pharmacol. 2020; 60:1573-1584.
44. Thurman J M, Marians R, Emlen W, Wood S, Smith C, Akana H, Holers V M, Lesser M, Kline M, Hoffman C, Christen E, Trachtman H. Alternative Pathway of Complement in Children with Diarrhea-Associated Hemolytic uremic Syndrome. Clin J Am Soc Nephrol. 2009; 4:1920-4.
45. Orth D, Khan A B, Naim A, Grif K, Brockmeyer J, Karch H, Joannidis M, Clark S J, Day A J, Fidanzi S, Stoiber H, Dierich M P, Zimmerhackl L B, Wurzner R. Shiga Toxin Activated and Binds Factor H: Evidence for an Active Role of complement in Hemolytic Uremic Syndrome. J Immunol. 2009; 182:6394-400.
46. Stahl G L, Xu Y, Hao L, Miller M, Buras J A, Fung M, Zhao H. Role for the alternative complement pathway in ischemia/reperfusion injury. Am J Pathol. 2003 February; 162 (2): 449-55.
47. Rother R P, Rollins S A, Mojcik C F, Brodsky R A, Bell L. Discovery and development of the complement inhibitor eculizumab for the treatment of paroxysmal nocturnal hemoglobinuria. Nat Biotechnol. (2007) 25:1256-64.
48. Pierangeli S S, Girardi G, Vega-Ostertag M, Liu X, Espinola R G, Salmon J. Requirement of activation of complement C3 and C5 for antiphospholipid antibody-mediated thrombophilia. Arthritis Rheum. 2005; 52 (7): 2120-2124.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a thrombotic microangiopathy (TMA) in a subject, the method comprising administering to the subject a therapeutically effective amount of an antibody that binds to and inhibits MBL2 and an antibody that binds to and inhibits FXIa.

2. The method of claim 1, comprising administering a pharmaceutical composition that comprises both an antibody that binds to and inhibits MBL2 and an antibody that binds to and inhibits FXIa in a single composition.

3. The method of claim 1, comprising administering a first pharmaceutical composition that comprises an antibody binds to and that inhibits MBL2 and a second pharmaceutical composition that comprises an antibody that binds to and inhibits FXIa, in a separate composition.

4. The method of claim 1, wherein:
the antibody that binds to and inhibits MBL2 is 3F8 or an analogue thereof, and/or,
the antibody that binds to and inhibits FXIa is 3G3 or an analogue thereof.

5. The method of claim 1, wherein the antibodies are human or humanized.

6. The method of claim 1, wherein the subject has enterohemorrhagic hemolytic uremic syndrome (eHUS); atypical HUS (aHUS); myocardial ischemic reperfusion injury; renal ischemic reperfusion injury; stroke; eclampsia or pre-eclampsia; catastrophic antiphospholipid antibody syndrome; complement-related renal injury; hematopoietic stem cell transplant thrombotic microangiopathy; paroxysmal nocturnal hemoglobinuria (PNH); disseminated intravascular coagulation (DIC); or venous or arterial thrombosis.

7. The method of claim 6, wherein the stroke is arterial ischemic stroke or perinatal arterial ischemic stroke.

8. A pharmaceutical composition comprising as active agents an antibody that binds to and inhibits MBL2 and an antibody that binds to and inhibits FXIa.

9. The composition of claim 8, wherein:
the antibody that inhibits MBL2 is 3F8 or an analogue thereof, and/or,
the antibody that inhibits FXIa is 3G3 or an analogue thereof.

10. The composition of claim 8, wherein the antibodies are human or humanized.

11.-17. (canceled)
